(19) United States
(12) Patent Application Publication
(10) Pub. No.: US 2011/0245087 A1
(43) Pub. Date: Oct. 6, 2011

(54) METHOD FOR OPTIMIZING AND VALIDATING AN ASSAY FOR DETERMINING THE PRESENCE OR ABSENCE OF A MEDICAL CONDITION

(76) Inventor: Gunter Weiss, Berlin (DE)

(21) Appl. No.: 12/736,212

(22) PCT Filed: Mar. 18, 2009
(86) PCT No.: PCT/IB2009/005344

§ 371 Date: Sep. 20, 2010

(30) Foreign Application Priority Data
Mar. 18, 2008 (EP) 08007488.3
May 21, 2008 (EP) 08156654

(51) Int. Cl. C40B 30/00; C12Q 1/68

(52) U.S. Cl. 506/7; 435/6.11

(57) ABSTRACT

The invention relates to a method for validation of an assay for determining the presence or absence of a medical condition, wherein the nucleic acid has been treated such that all unmethylated cytosine bases are converted to uracils. According to the invention, the method comprises: a) measuring the concentration of the nucleic acid in biological samples; b) allotting the samples based on the measured concentration of the nucleic acid in the sample to a first sample group if the concentration of the nucleic acid is below a given threshold value, or to a second sample group if the concentration of the nucleic acid is above the given threshold value; c) performing an assay for determining the methylation status of the nucleic acid in the sample obtaining methylation signals, and d) applying a first algorithm to the value if the sample was allotted to the first sample group, or a second algorithm if the sample was allotted to the second sample group.

The present invention relates to a method for optimizing and validating an assay for determining the presence or absence of a medical condition based on the determination of the methylation status of a nucleic acid in a biological sample.

### BACKGROUND

Compared to a number of other diagnostic tools used for diagnosis of disease or disease states, molecular analysis bears the advantage of requiring only a very small amount of a patient's DNA and therefore being more time effective, less labour intensive and more convenient for the patient, as it is often sufficient to take a body fluid sample from the patient such as blood, or urine, compared to routinely used invasive methods such as biopsy taking or colonoscopy. Despite elevated costs, many clinical and public health diagnostic laboratories have implemented molecular techniques due to superior rapidity and accuracy in comparison with traditional methods. However, in order to become a reliable medical tool, which in the long run could make further invasive diagnosis superfluous, the diagnosis based on molecular tools should be as sensitive and as specific as methods that are routinely used at present.

When determining the validity of a diagnostic assay, various methods of determining the performance of an assay, i.e. its sensitivity and specificity, are known. However, under certain conditions, these methods have shown to undervalue the performance of particular assays.

### DESCRIPTION OF THE INVENTION

Accordingly, an object of the present invention was to provide a means for optimizing and validating an assay for determining the presence or absence of a medical condition based on the methylation status of a nucleic acid in a biological sample.

The inventor has surprisingly found that when using patient samples with varying nucleic acid concentrations, the known methods for determining the performance of a quantitative assay do not always yield a truthful picture of the assays performance.

In contrast, the present invention provides a method for optimizing and validating the relationship between sensitivity and specificity, i.e. the performance, of an assay for determining the presence or absence of a medical condition based on the methylation status of a nucleic acid in a biological sample. The method can be used for any assay performed on a nucleic acid that results in providing a binary result, such as the presence or absence of a nucleic acid parameter.

The assay will usually be based on a particular sequence in the nucleic acid contained in the sample, which can be of varying length. This sequence is referred to herein as the target sequence.

The term specificity is meant to refer to be a value describing the ratio of negative assay results in true negative samples. If amongst 100 true negative samples the assay stages 2 positive samples, the rate of false positives is 2% and the specificity is 98%. A high specificity of an assay therefore results in a low number of false positives.

The less specific the assay the higher is the number of false positives, wherein a false positive means a sample that was diagnosed as positive (i.e. the indicative sequence variation was detected (if the diagnosis is depending on presence or absence) or was detected to such an amount that it was interpreted as positive (if the diagnosis depends on a detected amount)) but where the sample is a true negative, i.e. the patient was diagnosed as normal, or healthy as compared to diseased by the gold standard.

The term sensitivity is meant to refer to be a value describing the ratio of positive assay results in true positive samples. If amongst 10 true positive samples the assay stages 8 positive, the rate of false negatives is 20% and the sensitivity is 80%. A high sensitivity of an assay results in a low number of false negatives, i.e. patients diagnosed positively with disease or disease condition by the gold standard and not diagnosed positive by the assay.

The method can e.g. be used for determining the presence or absence of a disease like cancer, wherein the diseased tissue is characterized by a particular single nucleotide polymorphisms (SNP) that is not present in healthy tissue. When performing a diagnostic assay from body fluid, the fraction of nucleic acids stemming from the cancerous tissue is small compared to the fraction of nucleic acids stemming from healthy tissues. The total nucleic acid concentration varies for each sample obtained from a patient, such as the fraction of nucleic acid stemming from the diseased tissue of total nucleic acid.

It was found that not all of the biological or patient samples are equally suitable for determining both sensitivity and specificity of a diagnostic assay when validating it. Instead, it was surprisingly found that grouping of the samples based on their nucleic acid concentration allows for improving the performance of a diagnostic assay in terms of both sensitivity and specificity when validating it.

A preferred embodiment of the method of the invention relates to a method for validation of an assay for determining the presence or absence of a medical condition based on the methylation status of a nucleic acid in a biological sample.

The methylation of cytosine residues of nucleic acid is a phenomenon that has been correlated with gene regulation. Certain cell types consistently display specific methylation patterns, and this has been shown for a number of different cell types (Adorjan et al. (2002) Tumor class prediction and discovery by microarray-based DNA methylation analysis. Nucleic Acids Res 30(5) e21).

In higher order eukaryotes DNA is methylated nearly exclusively at cytosine bases located 5′ to guanine in a CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females.

The modification of cytosine in the form of methylation contains significant information. The identification of 5-methylcytosine within a DNA sequence is of importance in order to uncover its role in gene regulation. The position of a 5-methylcytosine cannot be identified by a normal sequencing reaction, since it behaves just as an unmethylated cytosine as per its hybridization preference. Furthermore, in any standard amplification, such as a standard polymerase chain reaction (PCR), this relevant epigenetic information will be lost.

Several methods are known to solve this problem. Generally, genomic DNA is treated with a chemical or enzyme leading to a conversion of the cytosine bases, which consequently allows distinguishing between methylated and unmethylated cytosine bases. The most common methods are a) the use of methylation-sensitive restriction enzymes capable of differentiating between methylated and unmethylated DNA and b) the treatment with bisulfite. The use of methylation-sensitive restriction enzymes is limited due to the selectivity of the restriction enzyme towards a specific recognition sequence.

Therefore, the ‘bisulfite treatment’, allowing for the specific reaction of bisulfite with cytosine (which upon subsequent alkaline hydrolysis is converted to uracil, whereas 5-methylcytosine remains unmodified under these conditions (Shapiro et al. (1970) Nature 227: 1047)) is currently the most frequently used method for analyzing DNA for the presence of 5-methylcytosine. Uracil corresponds to thymine in its base pairing behavior, that is, it hybridizes to adenine. 5-methylcytosine does not change its chemical properties under this treatment and therefore still hybridizes with guanine. Consequently, the original DNA is converted in such a manner that 5-methylcytosine, which originally could not be distinguished from cytosine by its hybridization behavior, can now be detected as the only remaining cytosine using conventional molecular biological techniques, such as amplification and hybridization or sequencing. Comparing the sequences of the DNA with and without bisulfite treatment allows easy identification of those cytosines that have been methylated in vivo.

Accordingly, in such a preferred embodiment of the method of the invention, the nucleic acid used in the assay has been treated such that all unmethylated cytosine bases are converted to uracil bases. This kind of nucleic acid is known to be useful for determining the methylation status of a nucleic acid, in particular of genomic nucleic acid. In such a case, the binary result in the presence or absence of a particular methylation at a particular cytosine residue or a set of cytosine residues of the nucleic acid analyzed.

The conversion of all unmethylated cytosine bases to uracils can be performed by different methods, such as a conversion based on a treatment of the nucleic acid with bisulfite or based on an enzymatic treatment. Bisulfite conversion is preferred. In converted nucleic acid, in particular DNA or genomic DNA, all unmethylated cytosines are converted to uracils and all methylated cytosines remain cytosines, rendering methylated and unmethylated cytosine residues distinguishable from each other.

Usually, a first study is performed with a diagnostic assay that is to be validated in order to determine which sensitivity and specificity can and should be achieved. Based on this information, a second study is performed to validate the assay.

The invention is based on performing not only the diagnostic assay to be validated on the biological samples, but also determining the nucleic acid concentration of each sample. Based on the latter, the samples are divided into groups. The methylation value obtained by the assay for each biological sample is then subjected to an algorithm for determining the methylation result, whereby the kind of algorithm that is applied depends on which sample group the sample belongs to. This way, the performance of the assay is optimized.

Specifically, the method of the invention comprises the following steps:

a) The concentration of the nucleic acid in a multitude of biological samples is measured. This can be done either before or after conversion, i.e. either with treated or untreated nucleic acid. Usually, the biological samples will be patient samples. The concentration measurement can be performed using any suitable method. It is however preferred to use real-time PCR to measure the nucleic acid concentration. A preferred assay for determining the concentration of bisulfit-converted nucleic acid is the HB14 assay. The CCF1 assay is preferred for determining the concentration of genomic DNA. Both of theses assays are known to a person of skill in the art. The multitude of biological samples is the samples that are used to validate the diagnostic assay in a study.

b) The samples are allotted to samples groups based on the measured concentration of the nucleic acid in each of the samples. In particular, if the measured concentration of the nucleic acid in a sample is below a given threshold value, the sample is allotted to a first sample group. If the measured concentration of the nucleic acid in a sample is at or above a given threshold value, the sample is allotted to a second sample group. Alternatively, if the measured concentration of the nucleic acid in a sample is below or at a given threshold value, the sample is allotted to a first sample group, and if the measured concentration of the nucleic acid in a sample is above a given threshold value, the sample is allotted to a second sample group. As will be described in more detail below, both the first and the second sample group will be used for determining the performance of the diagnostic assay, but based on different algorithms that are used to determine whether a sample is classified as positive (disease present) or negative (disease absent).

The term diagnostic assay includes assays that are used to predict the progression of a disease or condition of a subject or patient.

c) For each sample, an assay for determining the methylation status of the nucleic acid in the sample at least twice in independent experiments for obtaining at least two methylation values (the binary result, as explained above, i.e. either the methylation is present in the nucleic acid or absent). In order to perform independent experiments, the sample may be divided into sub-samples so that each of the sub-samples is subjected to an assay or the first and the second assay are performed in a multiplex fashion simultaneously.

d) One of at least two algorithms is applied to the methylation results that were obtained. Which algorithm is applied depends on whether the sample was allotted to the first or to the second sample group. Specifically, a first (or sensitivity) algorithm is applied to the value for determining the performance of the assay if the sample was allotted to the first sample group. If the sample was allotted to the second sample group, a second (or specificity) algorithm is applied to the value in order to determine the performance of the assay. In other word, a first or sensitivity algorithm is applied to the methylation value of each of the samples of the first sample group for determining a methylation result of the applied assay (assay result), and/or a second algorithm (specificity algorithm) is applied to the methylation value of the samples of the second sample group for determining an assay result. The assay result can be understood as the end result of the present method, i.e. whether the biological sample is regarded as positive (having a methylated target nucleic acid) or negative (having an unmethylated target nucleic acid). The application of the first and/or second algorithm allows for determining the performance of the assay.

The algorithms are determined empirically, as understood by a person of skill in the art together with the examples provided herein. The exact algorithm depends e.g. on the number of independent experiments performed for each sample to determine the methylation value (i.e. methylation present or absent). When three independent experiments are performed, the first algorithm for high sensitivity can be “if at least 1 methylation assay out of 3 is positive, then the assay result of the assay is considered positive” and the second algorithm for high specificity can be “if at least 2 methylation assays out of 3 are positive, then the assay result is considered positive”.

If four independent experiments are performed for each sample, it needs to be empirically determined whether the first algorithm should be “if at least 1 methylation assay out of 4 is positive, then the assay result is considered positive” or rather “if at least 2 methylation assay out of 3 are positive, then the assay result is considered positive”. A person of skill in the art will know how to determine a suitable algorithm based on data from a first study performed with a multitude of biological samples.

e) The assay and the interpretation of its results is validated by determining whether the sensitivity and the specificity of the assay reaches given values.

The order of the above described steps may vary, where suitable, as evident for a person of skill in the art. For example, the measurement of the concentration of the nucleic acid can be performed before, after or simultaneously with the performance of the methylation assay.

Preferred embodiments of the invention are described with reference to the dependent claims.

Preferably, the threshold value used to determine whether a sample is allotted to the first or the second sample group is chosen such that a first fraction of the multitude of samples is allotted to the first sample group and a second fraction of the multitude of samples is allotted to the second sample group. The first fraction is preferably 75% and the second fraction is accordingly 25%, but any other distribution is also possible, as long as no fraction is 0%.

In another preferred embodiment of the method of the invention, the samples are also allotted to a third sample group based on the measured concentration or amount of the nucleic acid in the sample, namely if the measured concentration of the nucleic acid is below a given minimum threshold value. Samples that are allotted to this third sample group are considered not to contain nucleic acid are therefore not used for assay validation.

It is preferred that the biological sample that the assay to be validated is based on stems from a body fluid, because in such assay the problem of varying nucleic acid concentration is particularly prominent. The term body fluids is meant to comprise fluids such as whole blood, blood plasma, blood serum, urine, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctiva fluid, vaginal fluid, duodenal juice, pancreatic juice, bile, stool and cerebrospinal fluid. It is especially preferred that said body fluids are whole blood, blood plasma, blood serum, urine, stool, ejaculate, bronchial lavage, vaginal fluid and nipple aspirate fluid. It is particularly preferred that the body fluid is plasma.

Preferably, the nucleic acid is DNA, in particular genomic DNA.

The preferred means for making methylated cytosine residues distinguishable from unmethylated cytosine residues is through bisulfite. Bisulfite treatment can be performed with a bisulfite, a disulfite or a hydrogensulfite solution. As known to a person of skill in the art, the term “bisulfite” is used interchangeably for “hydrogensulfite”.

In a preferred embodiment of the method, the presence or absence of methylation is determined by means of an assay based on at least one of the following: array based assays, real-time assays, MSP, MethyLight, QM and/or HeavyMethyl. These methods are known to a person of skill in the art.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146. For analysis of methylation within nucleic acids subsequent to bisulfite treatment, it is required that the probe be methylation specific, as described in U.S. Pat. No. 6,331,393, also known as the MethyLight™ assay. The term “HeavyMethyl™” assay, in the embodiment thereof implemented herein, refers to an assay wherein methylation specific blocking probes (also referred to herein as blockers) covering CpG dinucleotides between, or covered by the amplification primers enable methylation-specific selective amplification of a nucleic acid sample.

In the nucleic acid or the sequence in the nucleic acid that the diagnostic assay to be validated is based on (target nucleic acid), the methylation status of at least one CpG or a group of CpGs is determined. This at least one CpG or a group of CpGs can be methylated in vivo and therefore be used for making a diagnostic prediction. The target nucleic acid can be a gene, including a regulatory sequence of a gene or promoter, or a sequence that is part of any of the beforementioned structures, in other words any nucleic acid sequence in which a methylated cytosine residue may occur. Possible genes that can be used in the present method are selected from the group consisting of Septin 9; SND1; PCDHGC3; EDNRB; STOM; GLI3; RXFP3; RASSF2; Q8N2B6; PCDH10; LIMK1; TFAP2E; PTGER4; SHOX2; RASSF2A; CCND2; RASSF1A; MSF; PRDM6; LMX1A; NR2E1; SCGB3A1; TMEFF2; NGFR; SLIT2 and/or DAPK1. Preferably, the target nucleic acid gene is a gene selected from the group consisting of Septin-9, TMEFF2, NGFR or a fragment of any of these. Possible methylation markers are also known from WO 2006/113466.

The medical condition that the assay to be validated is predicting is selected from the group consisting of cancers, solid tumors and cell proliferative disorders. The cancer can is preferably colon cancer, lung cancer or prostate cancer. The biological sample may be any suitable sample type comprising DNA including but not limited to cell lines, biopsies, blood, sputum, stool, urine, cerebral-spinal fluid, tissue embedded in paraffin such as tissue from eyes, intestine, kidney, brain, heart, prostate, lung, breast or liver, histological object slides, or combinations thereof.

Suitable assays for detecting methylation are known in the art. According to the present invention said assay may be a methylation sensitive restriction enzyme assay and/or a bisulfite based assay. These include but are not limited to array based assays, Real-Time amplification assays, MSP, MethyLight, QM or, particularly preferred, HeavyMethyl. It is particularly preferred that said assay is a quantitative assay, such as but not limited to Real-Time PCR based assays.

### FIGURES

FIGS. 1 to 4 show procedures and data described under examples below.

FIG. 1

Outline of the Septin 9 assay workflow. The assay was optimized for an input volume of 4-5 ml of plasma. Extraction of DNA from plasma and purification of DNA following bisulfite treatment used magnetic particle methods. Assay results could be reported within 32 hours from the start of sample processing.

FIG. 2

The Septin 9 real time PCR. (A) Target sequence (SEQ ID NO 1) of the Septin 9 real time PCR FRET probe assay compared and (B) of the new Septin 9 hydrolysis probe assay (SEQ ID NO 2). CpG sites are indicated by bold letters. Lower case letters “a” and “t” indicate positions of bisulfite conversion. The sequence in common between the assays is indicated by a box. Primer binding sites are indicated with solid arrows, the blocker binding regions are indicated with dashed lines and the probe binding sites are indicated with dash-dot lines. The blocker incorporates a tetrahydrofuran abasic nucleotide indicated in the sequence with an X, and has a 3′ C3 spacer to prevent extension. The selective amplification of methylated DNA is driven by binding of the blocker to the converted unmethylated sequence at the same CpG positions in both assays. The fluorescently labelled probes are methylation specific.

SEQ ID NO 1:
CCCACCAACC ATCATATCGA ACCCCGCGAT CAACGCGCAA
CTAAATAAAA TCATTTCGAA CTTCGAAAAT AAATACTAAA
CTAACTACTA C
SEQ ID NO 2:
GATTXGTTGT TTATTAGTTA TTATGTCGGA TTTCGCGGTT
AACGCGTAGT TGGATGGGAT TATTT

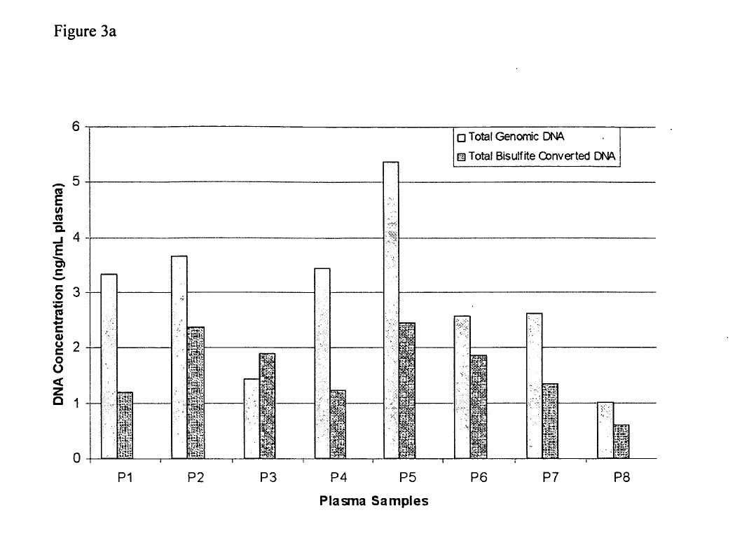

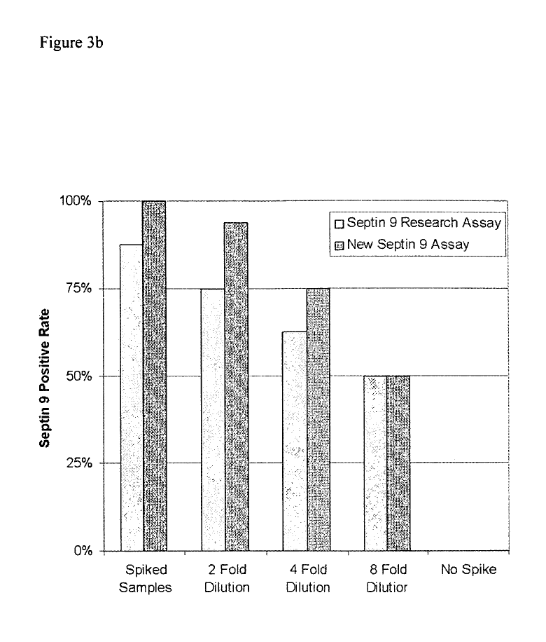

FIG. 3

Performance of the Septin 9 assay on model DNA samples.

FIG. 3a)

Concentration of total genomic DNA (gray bars) based on the CFF1 PCR assay, and bisulfite converted DNA (hatched bars) based on the β-actin PCR assay, calculated per ml of input plasma for eight independent sample pools. Samples were produced by spiking plasma positive for Septin 9 into Septin 9 negative plasma.

FIG. 3b)

Septin 9 positive rate in percentage measured for the Septin 9 FRET based research assay (gray bars) and the new Septin 9 assay (hatched bars). Plasma samples were prepared in a dilution series of Septin 9 positive plasma spiked into a background of Septin 9 negative plasma, with a target of less than 10 pg/ml in the 8 fold dilution samples. Each percentage measurement is the aggregate of PCR results for 8 independent spiked DNA pools at the given dilution.

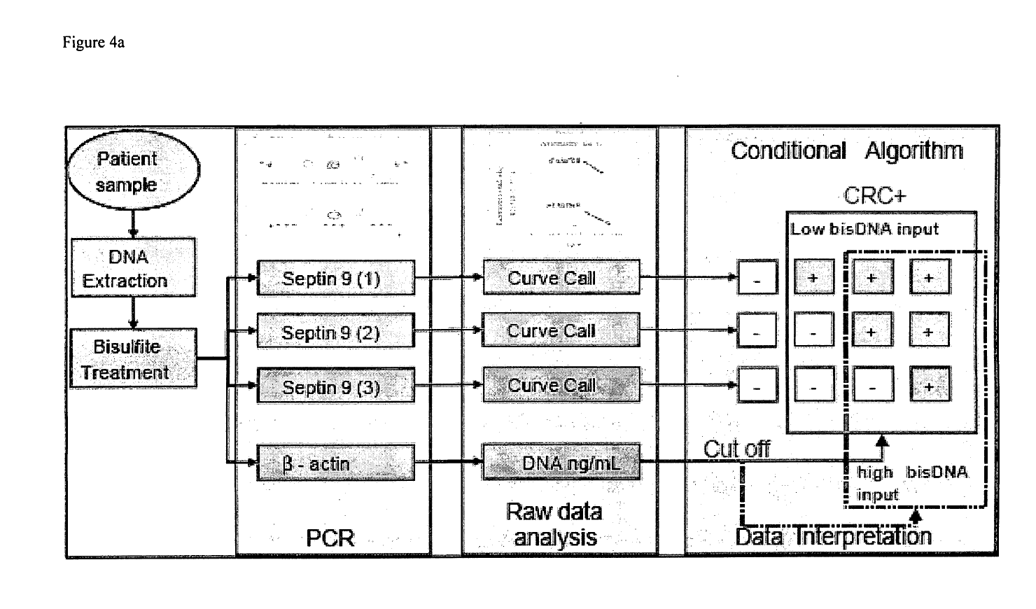

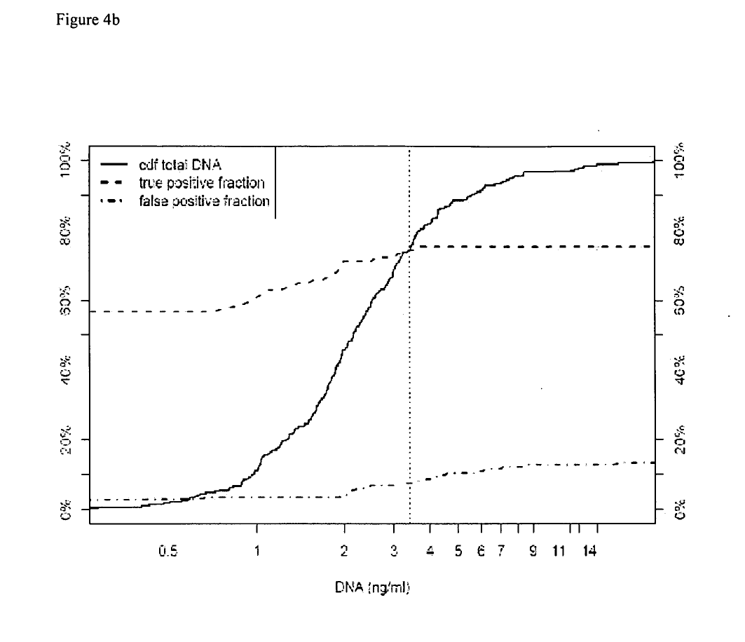

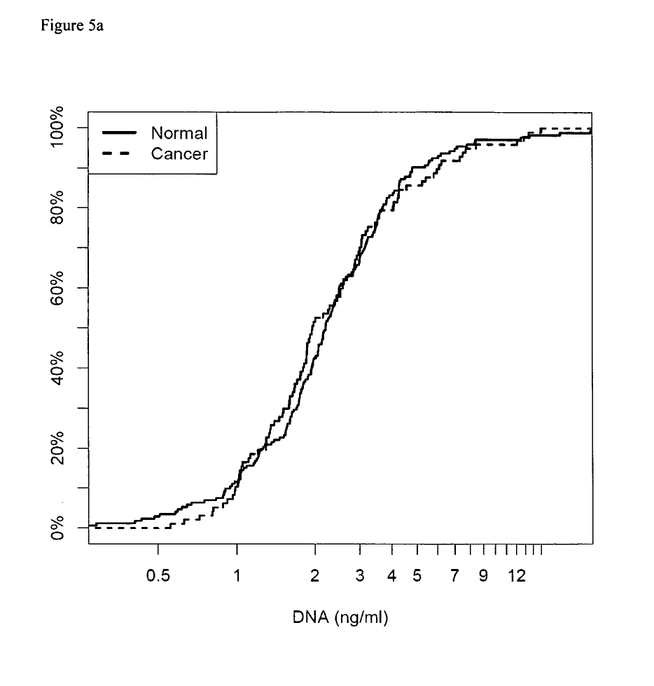

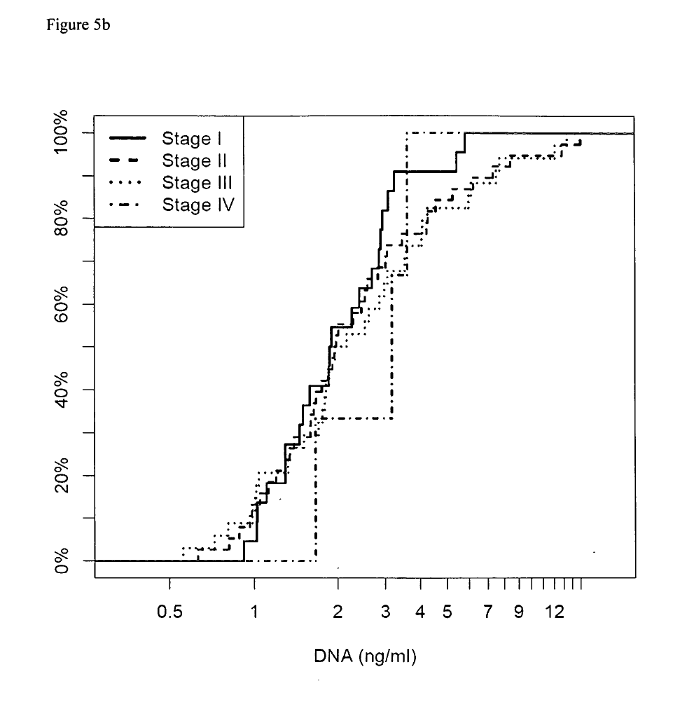

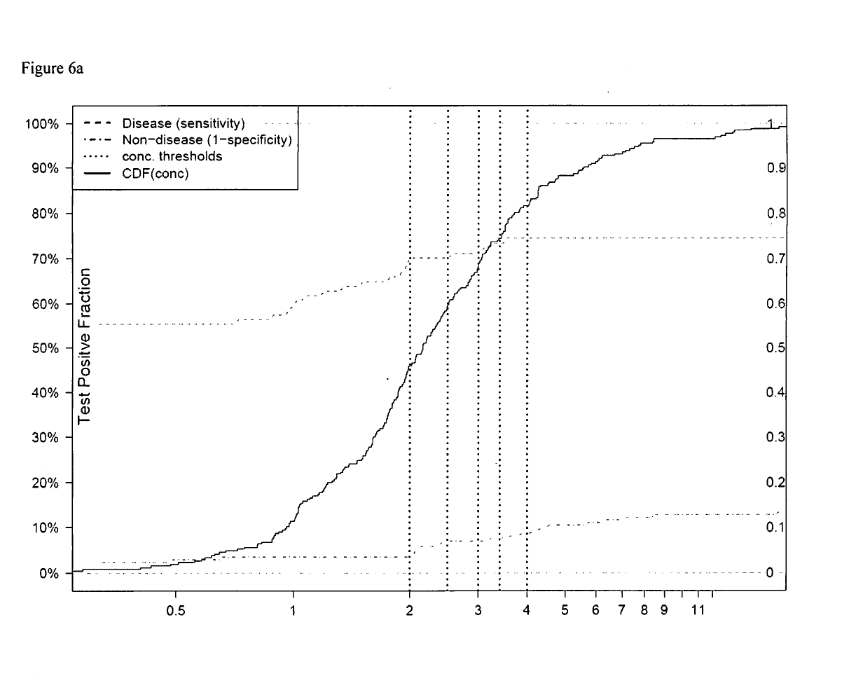

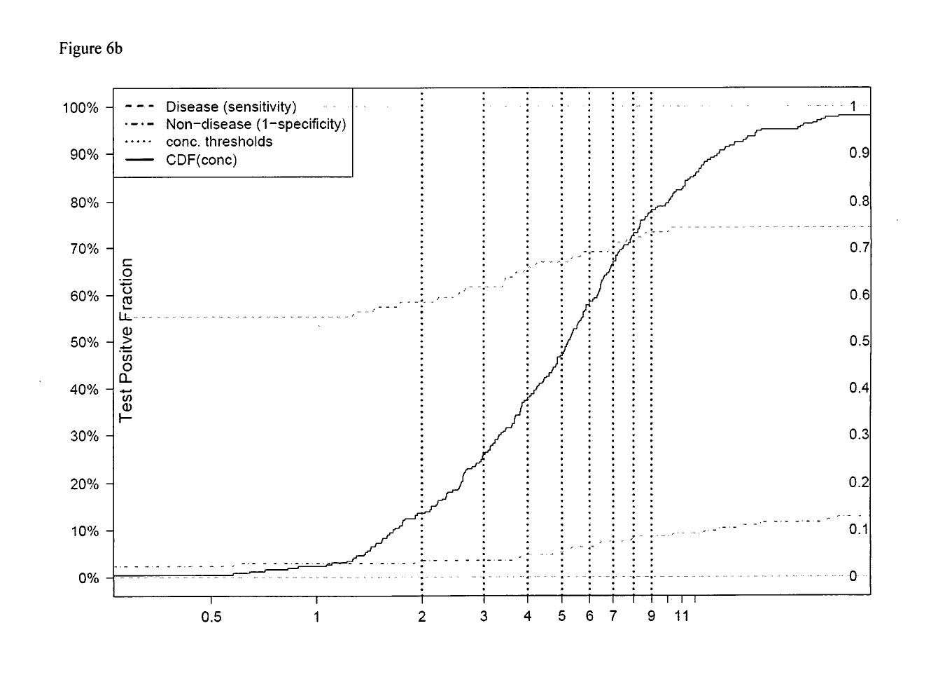

FIG. 4

An embodiment of the invention is shown, the Septin 9 “conditional qualitative algorithm”.

FIG. 4a)

Outline of an embodiment of the method of the invention, the Septin 9 “conditional qualitative algorithm”. The total DNA concentration following conversion through bisulfite treatment is measured for each sample using a β-actin PCR and based on a threshold (“cut off”, 3.4 ng/ml), samples are categorized for Septin 9 analysis, i.e. allotted to a first or second sample group. Samples with total DNA concentrations below the threshold are analyzed with a first algorithm, the high sensitivity criteria (at least one of three calls positive), and samples with DNA concentrations above the threshold are analyzed with a second algorithm, the high specificity criteria (at least two of three calls positive).

FIG. 4b)

Septin 9 detection as a function of total DNA recovery for the Training Set Data. The solid line indicates the cumulative distribution of the total DNA concentration in ng/mL on the X axis. The dotted vertical line indicates the selected decision point on the total DNA concentration scale (3.4 ng/ml). True (dashed line) and false positive (dashed dotted line) fractions are displayed as a function of the decision point on the total DNA concentration scale. The performance of the “high specificity” rule is represented as the percentage (Y axis) where the lines cross the left side of the chart, that of the conditional rule is indicated at the vertical line, and the “high sensitivity” rule performance where the lines cross the right side of the chart. The decision point was selected to optimize the true positive fraction, while minimizing the false positive fraction.

FIG. 5

FIG. 5a)

Plot of cumulative total bisulfite converted DNA measured by β-actin real time PCR, for cancer cases and control patients expressed in ng/ml plasma. The total DNA measurement is essentially identical for controls and cases.

FIG. 5b)

Plot of cumulative total bisulfite converted DNA measured by β-actin real time PCR, for cancer cases by stage in ng/ml plasma. The total DNA measurement is essentially identical for stages I-III but a number of stage IV cancers show high concentrations.

FIG. 6

FIG. 6a)

displays the example of Table 5 with an exemplary set of thresholds. For each threshold (five vertical lines) the respective parameters (sensitivity and 1-specificity) can be read off from the respective graphs.

FIG. 6b)

displays the example of Table 6 with an exemplary set of thresholds. For each threshold (eight vertical lines) the respective parameters (sensitivity and 1-specificity) can be read off from the respective graphs.

### EXAMPLES

The development of a new assay for the detection of SEPT9 methylation, and its validation as a colon cancer biomarker in two independent sample sets is described. The performance of this new Septin 9 assay in a new prospectively collected case control patient set and subsequent verification with a new independent set of cases and control samples is described.

### Materials and Methods

### Study Participants

Ethics review boards at collection sites approved study protocols, and all participants in the study provided written informed consent following local ethics requirements. The study included patients with all stages of colorectal cancer and individuals without diseases of the colon as verified by colonoscopy. The disease status and staging of colorectal cancer patients was obtained from pathology reports. All study participants were at least 37 years old, with a majority of patients being 50 and older. Participating subjects did not have a personal history of HIV, HBV or HCV or cancer other than basal cell skin cancer, nor symptoms of severe acute or exacerbated chronic disease. Collection of Plasma Blood samples were collected by phlebotomy using lavender topped EDTA Vacutainer tubes (BD Medical Systems, Franklin Lakes, N.J., USA). Plasma was prepared from blood samples within 4 hrs of collection by centrifugation of blood tubes (1500×g, 10 min), transferred to a 15 ml tube and centrifuged a second time (1500×g, 10 min). When transferring plasma, care was taken not to transfer buffy coat cells. Following the second centrifugation, all plasma from a given patient was pooled, aliquoted into cryovials and stored at −80° C. Samples were shipped to Epigenomics Inc. on dry ice, and stored at −80° C. until processed in the study. Surrogate samples: For workflow development two types of surrogate sample were produced: 1) purified methylated DNA (CpGenome, Millipore, Mass., USA) was spiked into plasma negative for Septin 9, 2) plasma positive for Septin 9 was spiked into plasma negative for the Septin 9 biomarker.

### Measurement of Septin 9

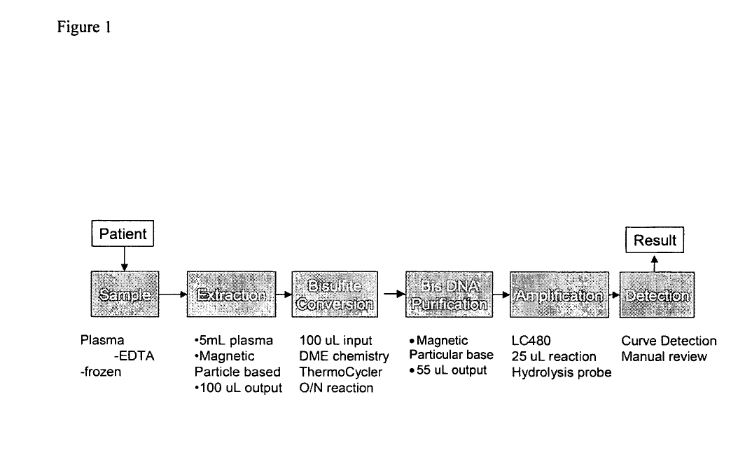

The workflow, which was developed for an input volume of 4-5 ml of plasma, consists of DNA extraction from plasma, bisulfite conversion of DNA, purification of converted DNA, and real time PCR as outlined in FIG. 1.

The extraction of circulating plasma DNA was based on a magnetic particle method, using a modified version of the 4.8 ml chemagic Viral DNA/RNA kit (chemagen AG, Baesweiler Germany). DNA from 4-5 ml of plasma was eluted in 100 μl of elution buffer, a 5 μl aliquot of which was used to measure total DNA recovery by real time PCR. For bisulfite conversion, bisulfite salt solutions, organic solvent (DME) and radical scavenger were added to the eluted DNA in a 0.5 ml elution tube, and the conversion was performed on an Eppendorf Mastercycler (Eppendorf, Hamburg Germany) for 7 hours at 50° C. with 3 thermal spikes at 99° C. Following the conversion reaction, DNA was purified using a magnetic particle based purification kit for bisulfite converted DNA (chemagen AG). Purified bisulfite converted DNA was eluted in 55 μl of final elution buffer (10 mmol/l Tris pH 7.2), and was used directly in real time PCR analysis. The oligonucleotide sequences and assay conditions for the CFF1 (total DNA), β-actin (total bisulfite converted DNA) and Septin 9 real time PCR assays used in this study are provided in Table 5. Real time PCR analysis was performed on the Lightcycler LC480 (Roche Applied Science, Basel Switzerland) using 96 well reaction plates, and the Quantitect Multiplex PCR mastermix (Qiagen, Venlo The Netherlands). Oligonucleotide quality was determined in house by MALDI-TOF prior to use, and the limit of detection for each PCR was evaluated prior to use in studies.

### Quality Control

The study was run in a batch mode, using positive and negative controls for each extraction and bisulfite batch. Positive controls for DNA extraction were 25 ng/ml CpGenome methylated DNA diluted in 5 mg/ml bovine serum albumin (BSA), while negative extraction controls were BSA without spiked DNA. Positive controls for bisulfite processing were composed of 10 ng of fully methylated CpGenome DNA spiked in 90 ng of human genomic DNA prepared from buffy coat cells (Roche Applied Sciences, Basel Switzerland) in a 100 μl volume of elution buffer, while negative bisulfite conversion controls were composed of elution buffer alone. Samples for the study were considered valid when the total bisulfite converted DNA measured by β-actin was >0.001 ng/ml. Statistical Analysis Data collection included total genomic DNA recovery following extraction, total bisulfite DNA recovery, three measurements of Septin 9 on undiluted samples and one measurement of Septin 9 on a 10 fold diluted sample. Prior to unmasking the sample identity, all PCR results were confirmed by visual inspection of the PCR curves. Each PCR run included a set of calibrator samples and at least three no template control samples. DNA concentration was determined from calibration curves by linear regression of crossing point values using the second derivative method (17). Samples with less than 0.001 ng/ml bisulfite converted DNA (based on the β-actin assay) (minimum threshold) were excluded from analysis.

### Study Design

For validation of the assay using clinical samples, a training and testing study design was followed. For the training study, plasma samples from 100 cancer cases, primarily Stage I-III, and from 175 non-cancer controls were processed and analyzed. The resulting data was analyzed using multiple algorithms to calculate optimized sensitivity and specificity values. In the test phase of the study, an independent sample set comprising 100 cancer cases and 170 non-cancer controls were blinded, processed using the training study workflow, and analyzed using the algorithms agreed to a priori based on the analysis of the training study.

### Results

### The Septin 9 Assay

In this study a new assay for SEPT9 promoter methylation, outlined in FIG. 1, was introduced which was developed to improve parameters critical for implementation in a standard molecular diagnostics laboratory. As indicated in FIG. 1, the protocol is designed for analysis of plasma collected using standard EDTA collection tubes. During sample preparation, care was taken when transferring plasma to avoid buffy coat cells, and a second centrifugation was added to clear the plasma further prior to freezing. Since careful preparation of plasma reduces background DNA resulting from cellular lysis during processing, reducing the variability in sample collection (18). In developing this protocol, PPT barrier tubes (Becton Dickenson, N.J., USA) were also tested, but observed elevated total DNA levels, likely due to incomplete clearing of blood cells (data not shown).

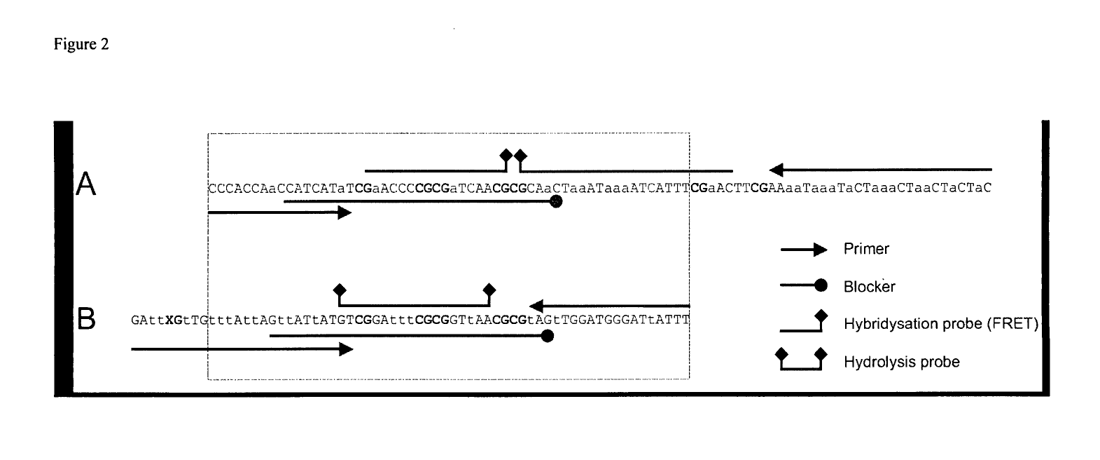

The assay procedure consists of the following steps:
extraction of circulating genomic DNA from plasma,
bisulfite conversion of the extracted DNA,
purification of bisulfite treated DNA, and
measurement of Septin 9 status by a real time PCR assay (FIG. 2).

In developing the Septin 9 test, our objective was to produce an optimized integrated assay by: 1) maximizing plasma input to increase sensitivity; 2) replacing the multiple parallel DNA extractions with a single extraction; 3) replacing purification by ultrafiltration with magnetic particle procedures; 4) reducing the final elution volume to increase DNA concentration; 5) extracting total genomic DNA including high and low molecular weight; 6) reducing the size of the real time PCR amplicon; 7) overcoming PCR inhibition; 8) providing an approach to carry-over prevention; 9) reducing cost and 10) increasing throughput. Assay procedures were developed using surrogate samples that included purified DNA, purified DNA spiked into Septin 9 negative plasma and Septin 9 positive plasma spiked into Septin 9 negative plasma. The procedures were then validated using clinical samples in the case control studies.

### Genomic DNA Extraction

The extraction procedure was developed for optimal isolation of a broad range of fragment sizes of circulating DNA from 4-5 ml of plasma. Several approaches were tested including plasma pre-concentration, fluid:fluid extraction, and multiple types of magnetic particles (data not shown) with the optimal method being a single extraction protocol based on the chemagic Viral RNA/DNA protocol (chemagen AG). The commercially available protocol was modified by chemagen AG to improve the binding of small fragmented DNA while retaining binding of high molecular weight DNA. Optimal binding and washing conditions were determined and established the 100 μl elution volume. To determine the performance of DNA extraction from plasma, total genomic DNA recovery was measured using the genomic real time PCR assay CFF1. An example experiment from the assay development process using spiked plasma samples is shown in FIG. 3. The recovery of total genomic DNA is shown for eight individual samples, in which average DNA recovery of 2.93±1.36 ng/ml of input plasma was measured from a set of surrogate plasma samples in which low levels of plasma containing methylated SEPT9 were spiked into a Septin 9 negative plasma background (FIG. 3a). Based on these and additional studies (not shown) equivalent recovery of genomic DNA to that measured with the research assay was demonstrated. This was confirmed in the training and test set studies in which the median DNA concentration for all samples was 5.1 ng/ml and 3.61 ng/ml of input plasma, respectively.

### Bisulfite Treatment and Purification of Converted DNA

Our objectives for improving on the research assay bisulfite procedure were to increase throughput by performing the DNA denaturation and conversion in a thermal-cycler and to support automation by magnetic particle based purification of the bisulfite converted DNA. To reduce the reaction volume sufficiently to use the thermal-cycler, the organic solvent dioxane was replaced with diethyleneglycoldimethylether (DME) which enabled us to reduce the reaction volume from 600 μl to 320 μl. A variety of magnetic particle systems were tested for purification of bisulfite converted DNA (data not shown) of which a protocol based on components developed by chemagen AG was optimized. The protocol was tested using surrogate samples as described for genomic DNA extraction, and as illustrated in the sample experiment in FIG. 3a, the recovery of bisulfite converted DNA measured using the real time β-actin PCR reaction was 1.6±0.63 ng/ml of starting plasma, a yield in the range of 55% of the total genomic DNA. This performance was confirmed in the training and testing studies where the observed median values were 2.2 ng/ml (1.5, 3.4) and 1.9 ng/ml (1.3, 3.0) of input plasma, respectively. An additional benefit was observed in that the rate of sample drop-out observed was only 1.9% in the training set and 2.4% in the test set, a considerable reduction compared with the ultrafiltration method for post bisulfite purification in the research assay. An additional benefit of the new post-bisulfite purification protocol is that by omission of the de-sulfonation step, the sulfonated elution product is resistant to UNGase activity supporting the potential for UNGase based carry-over prevention.

### Septin 9 Real Time PCR

In previous studies, a real time PCR assay for the measurement of Septin 9 that uses a blocker oligonucleotide to suppress the amplification of unmethylated target sequences, and fluorescence resonance energy transfer (FRET) MethylLight detection probes (11, 19) was introduced. In the current study, a modified Septin 9 real time PCR assay that produces a 65 nucleotide amplicon compared with the 91 nucleotide research assay and uses five of the six CpG positions interrogated in the original assay was developed (FIG. 2). The specific sequences and reaction conditions are reported in Table 4. Use of a hydrolysis probe allowed to shorten the probed sequence within the amplicon, and by designing the blocker oligonucleotide to overlap the probe binding site (FIG. 2) the amplicon size could be reduced. The shortened assay seems to improve sensitivity by increased detection of methylated SEPT9 in fragmented DNA. The performance of the Septin 9 PCR measured as Limit of Detection (LOD) for methylated DNA spiked into an unmethylated background was 9.4 pg, representing a relative detection rate of at least 1:5000 (FIG. 5).

### Technical Evaluation of the Septin 9 Assay

The performance of the new assay was compared with the research assay in a study using surrogate samples in which Septin 9 positive plasma was spiked in a dilution series into a background of Septin 9 negative plasma (FIG. 3b) with the target concentration of Septin 9 biomarker at less that 10 pg/ml in the 8 fold dilution samples. For each dilution, the PCR positive rate for the two assays was measured in 8 independent spiked samples. The detection rates differed marginally between the two assays at the higher concentrations of the Septin 9 biomarker, and were identical (50%) at the greatest dilution (FIG. 3b). Based on these results and numerous additional experiments (data not shown), for surrogate samples the new assay essentially equivalent to the previously described research assay was considered, and it was proceeded to validate the assay with clinical samples in a training and test study.

### Clinical Case Control Studies

Having established the technical performance of the Septin 9 assay, assay performance in two prospectively collected case control studies were verified following a training/testing study design. In the training study, plasma samples were collected from 100 colorectal cancer cases and 175 colonoscopy verified non-cancer controls. Three case samples and two control samples were excluded from data analysis as a result of having less than 0.001 ng/ml bisulfite converted DNA (based on the β-actin real time PCR), i.e. the nucleic acid concentration was below the minimum threshold value. A sixth patient was excluded due to a corrected diagnosis of adenomatous polyp resulting in a final training sample set of 97 cases and 172 controls (Table 1). In the testing study, plasma from 100 cases and 170 controls were collected of which 6 cases and 13 controls were invalid due to a batch processing error and 4 cases and 2 controls were excluded as a result of having less than 0.001 ng/ml bisulfite DNA, i.e. the nucleic acid concentration was below the minimum threshold value. Thus the final testing sample set consisted of 90 cases and 155 controls (Table 1). For both the training and test studies, the collection on Stage I-III cancer was focused on, but with updated staging information, three cases were corrected to Stage IV in the training study, and 4 cases were corrected to Stage IV in the testing study. In both the training and test studies, total extracted genomic DNA and total bisulfite converted DNA was measured with single measurements. The Septin 9 assay was performed in triplicate on undiluted samples. Measurement of a 1:10 dilution of all samples showed no evidence for PCR inhibition with the new Septin 9 assay. Measurement of total DNA showed no significant difference between non-cancer controls and Stage I-III cancer cases while higher DNA concentrations were observed in some Stage IV cancer cases (FIG. 5a, 5b).

### Training Study

To optimize the performance of the Septin 9 assay, the data with several algorithms as reported in Table 2 was analyzed. For qualitative analyses, a sample was scored as positive or negative for Septin 9 by review of the amplification curves. For high sensitivity, samples were considered positive when at least one of three Septin 9 PCR reactions were positive. As shown in Table 2, the performance for all patients was 75% (65%; 83%), while for Stage I-III sensitivity was 74% (64%; 83%), at a specificity of 87% (81%; 91%). For high specificity analysis, samples were counted as Septin 9 positive if at least two of three curve calls were positive, resulting in an overall sensitivity of 57% (46%; 67%), with a sensitivity of 55% (45%; 66%) for Stage I-III and an improved specificity of 98% (94%; 99%).

A third approach to analysis, the method of the invention was used which combined the quantitative measurement of total DNA and the qualitative analysis of Septin 9 (FIG. 4a). It was observed that the false positive rate in the high sensitivity mode increased with increasing total DNA concentration (FIG. 4b) and it was reasoned that a total DNA threshold value could be establish below which a single positive curve was sufficient for a positive call (high sensitivity interpretive criteria), and above which at least two positive curves were required for a positive call (high specificity interpretive criteria). The third quartile bound of the β-actin measurement was selected as a threshold DNA concentration (3.4 ng/ml) such that 75% of the samples were analyzed according to the high sensitivity interpretive criteria, and 25% were analyzed according to high specificity criteria. This optimized conditional algorithm resulted in a sensitivity of 73% (63%; 82%) for Stage I-III cancers and a specificity of 92% (87%; 96%) (Table 2). Application of the conditional algorithm resulted in 10 false positive control samples being properly classified as negative, improving the specificity, and only one positive cases being reclassified as negative.

### Testing Study

The Test Set comprised 90 valid cancer samples and 155 non-cancer controls. The samples were processed in a masked manner, and the results recorded based on the algorithms established in the training set. The sample key was unmasked on completion of the study and the results summarized in Table 3. For Stages I-III, a sensitivity of 71% (60%; 80%) at a specificity of 86% (79%; 91%) in the high sensitivity analysis, 55% (44%; 65%)/95% (91%; 98%) in the high specificity analysis and 67% (56%; 77%)/89% (83%; 93%) in the conditional qualitative analysis was observed.

### Discussion

It was demonstrated that for early stage colorectal cancer (I-III), specific biomarkers are essential for cancer detection, and that in contradiction with some reports (26), total plasma DNA concentration is not a useful biomarker (FIG. 5a, 5b). For all analyses, samples below the minimum threshold, i.e. having less than 0.001 ng/ml converted DNA are considered invalid measurements, since the absence of DNA is indicative of a technical failure of the assay.

For valid samples, the Septin 9 results could be calibrated to maximize sensitivity (a single positive replicate is scored positive), or maximize specificity (two or three replicates are required to be positive for a positive call).

In the training study, a sensitivity of 74% at a specificity of 87% using the high sensitivity criteria, and a sensitivity of 55% at a specificity of 98% using the high specificity criteria was observed. In reviewing the false positive non-cancer controls results, that a correlation with increased DNA concentration was surprisingly observed.

On this basis, the method of the invention was developed in which a DNA recovery threshold was used to assign samples to either high specificity or high sensitivity interpretive criteria. Using this method of the invention, herein also called “conditional qualitative analysis”, a sensitivity of 73% at a specificity of 92% in the training study was observed. Applying this algorithm in the blinded testing study, a sensitivity of 67% at a specificity of 89% was observed. The results for both the training and test studies corroborate the previous findings in which it was reported that methylation of the promoter region of SEPT9 was highly correlated with the presence of colorectal cancer (11, 13). The combined results of these multiple studies strongly support the potential for the use of Septin 9 as a biomarker for colorectal cancer.

TABLE 1
Disease and Stage Distribution of Patient Samples in the Training and Test Studies

| | Training Set Samples | | | | Test Set Samples | | | |
|---|---|---|---|---|---|---|---|---|
| Sample | | | | Median Age | | | | Median Age |
| Group | Total | Female | Male | (range) | Total | Female | Male | (range) |
| CRC (all) | 97 | 33 | 64 | 62.5 (37-87) | 90 | 39 | 51 | 65 (41-86) |
| Stage I | 22 | 5 | 17 | 64 (47-79) | 19 | 8 | 11 | 66 (53-82) |
| Stage II | 38 | 21 | 17 | 63 (37-87) | 40 | 19 | 21 | 66 (41-86) |
| Stage III | 34 | 6 | 28 | 60 (40-86) | 27 | 11 | 16 | 60 42-75) |
| Stage IV | 3 | 1 | 2 | 47 (45-62) | 4 | 1 | 3 | 66 (53-73) |
| Controls | 172 | 87 | 85 | 60 (40-87) | 155 | 91 | 64 | 54 (40-90) |
| Total | 269 | | | | 245 | | | |

TABLE 2
Training Set Results. The performance of the Septin 9 assay based on
different qualitative analyses of triplicate PCR reactions.

| | High | | | | Conditional | |
|---|---|---|---|---|---|---|
| | Sensitivity (⅓) | | High | | Qualitative | |
| | Posi- | | Specificity (⅔) | | Posi- | |
| Patient | tive/ | % | Positive/ | % | tive/ | % |
| Group | Tested | Positive | Tested | Positive | Tested | Positive |
| Stage I | 10/22 | 45 | 7/22 | 32 | 10/22 | 45 |
| Stage II | 32/38 | 84 | 25/38 | 66 | 31/38 | 82 |
| Stage III | 28/34 | 82 | 20/34 | 59 | 27/34 | 79 |
| Stage IV | 3/3 | 100 | 3/3 | 100 | 3/3 | 100 |
| Stage I-III | 70/94 | 74 | 52/94 | 55 | 68/94 | 72 |
| All CRC | 73/97 | 75 | 55/97 | 57 | 71/97 | 73 |
| Controls | 23/172 | 13 | 4/172 | 2 | 12/172 | 7 |
| (Specificity) | | (87) | | (98) | | (93) |

TABLE 3
Test Set Results. Qualitative analysis of the performance of the Septin 9
assay using the calling algorithm established in the training set and
applied to a blinded test set

| | High | | | | Conditional | |
|---|---|---|---|---|---|---|
| | Sensitivity (⅓) | | High | | Qualitative | |
| | Posi- | | Specificity (⅔) | | Posi- | |
| Patient | tive/ | % | Positive/ | % | tive/ | % |
| Group | Tested | Positive | Tested | Positive | Tested | Positive |
| Stage I | 10/19 | 53 | 5/19 | 26 | 9/19 | 47 |
| Stage II | 30/40 | 75 | 24/40 | 60 | 29/40 | 73 |
| Stage III | 21/27 | 78 | 18/27 | 67 | 20/27 | 74 |
| Stage IV | 4/4 | 100 | 3/4 | 75 | 4/4 | 100 |
| Stage I-III | 61/86 | 71 | 47/86 | 55 | 58/86 | 67 |
| All CRC | 65/90 | 72 | 50/90 | 56 | 62/90 | 69 |
| Controls | 22/155 | 14 | 7/155 | 5 | 17/155 | 11 |
| (Specificity) | | (86) | | (95) | | (89) |

TABLE 4
Oligonucleotide sequences, concentrations and cycling conditions for the real
time PCR assays used in the study described in this example (μM: μmol/l).

| | Forward | Reverse | | | Cycling |
|---|---|---|---|---|---|
| PCR | Primer | Primer | Blocker | Probe | Conditions |
| SEPTIN 9 | AAATAATCC | GATT-DS- | GTTATTATGTT | FAM- | Activation - 95° C. |
| ASSAY | CATCCAACT | GTTGTTTATTAG | GGATTTTGTGG | TTAACCGCGAAA | 30 min. |
| CONC. | A | TTATTATGT | TTAATGTGTAG- | TCCGAC-BHQ1 | 55 Cycles: 95° C. |
| | (SEQ ID NO 3) | (SEQ ID NO 4) | C3 | (SEQ ID NO 6) | 10 sec |
| | 0.3 μM | 0.3 μM | (SEQ ID NO 5) | 0.1 μM | (4.4° C./sec), 56° C. |
| | | | 1.0 μM | | 30 sec (2.2° C./sec) |
| | | | | | cooling - 40° C. |
| | | | | | 5 sec (2.2° C./sec). |
| CFF1 | TAAGAGTAA | CCTCCCATCTCC | N/A | 6FAM- | Activation - 95° C. |
| (GENOMIC | TAATGGATG | CTTCC | | ATGGATGAAGAA | 15 min. |
| ASSAY) | GATGATG | (SEQ ID NO 8) | | AGAAAGGATGA | 50 Cycles: 95° C. |
| ASSAY | (SEQ ID NO 7) | 0.63 μM | | GT-BHQ-1 | 10 sec |
| CONC. | 0.63 μM | | | (SEQ ID NO 9) | (4.4° C./sec), 58° C. |
| | | | | 0.2 μM | 60 sec (2.2° C./sec) |
| | | | | | cooling - 40° C. |
| | | | | | 5 sec (2.2° C./sec). |
| β-ACTIN | GTGATGGAG | CCAATAAAACC | N/A | FAM- | Activation - 95° C. |
| (BISULFITE | GAGGTTTAG | TACTCCTCCCTT | | ACCACCACCCAA | 30 min. |
| ASSAY) | TAAGTT | AA | | CACACAATAACA | 50 Cycles: 95° C. |
| ASSAY | (SEQ ID NO 10) | (SEQ ID NO 11) | | AACACA-BHQ1a | 10 sec |
| CONC. | 0.9 μM | 0.9 μM | | (SEQ ID NO 12) | (4.4° C./sec), 57° C. |
| | | | | 0.1 μM | 30 sec |
| | | | | | (2.2° C./sec), 72° C. |
| | | | | | 10 sec (4.4° C./sec) |
| | | | | | cooling - 40° C. |
| | | | | | 5 sec (2.2° C./sec). |

TABLE 5
Analyses of a set of 94 (CRC) patients
and 172 normal control individuals.

| quantitation threshold (ng/ml) | sensitivity | specificity | accuracy |
|---|---|---|---|
| 2 out of 3 (Algorithm A) | 0.553 | 0.977 | 0.827 |
| 0.32 | 0.553 | 0.977 | 0.827 |
| 0.40 | 0.553 | 0.977 | 0.827 |
| 0.50 | 0.553 | 0.971 | 0.823 |
| 0.63 | 0.553 | 0.971 | 0.823 |
| 0.79 | 0.564 | 0.965 | 0.823 |
| 1.00 | 0.596 | 0.965 | 0.835 |
| 1.26 | 0.628 | 0.965 | 0.846 |
| 1.59 | 0.649 | 0.965 | 0.853 |
| 2.00 | 0.702 | 0.965 | 0.872 |
| 2.51 | 0.702 | 0.93 | 0.85 |
| 3.16 | 0.723 | 0.93 | 0.857 |
| 3.40 | 0.734 | 0.924 | 0.857 |
| 3.98 | 0.745 | 0.913 | 0.853 |
| 5.01 | 0.745 | 0.895 | 0.842 |
| 6.31 | 0.745 | 0.884 | 0.835 |
| 7.94 | 0.745 | 0.878 | 0.831 |
| 10.00 | 0.745 | 0.872 | 0.827 |
| 12.59 | 0.745 | 0.872 | 0.827 |
| 15.85 | 0.745 | 0.872 | 0.827 |
| 19.95 | 0.745 | 0.866 | 0.823 |
| 25.12 | 0.745 | 0.866 | 0.823 |
| 31.62 | 0.745 | 0.866 | 0.823 |
| 1 out of 3 (Algorithm B) | 0.745 | 0.866 | 0.823 |

Column 1 shows examples of set thresholds of a HB14 quantitation of bisulfite-treated DNA. Column 2 provides the respective sensitivity estimates, Column 3 the respective specificity estimates, and Column 4 provides the respective accuracy estimates. Highlighted (bold) are exemplary thresholds that provide significant better performance when the method of the invention (concentration dependent algorithm) is applied. Depending on the main focus of the clinical application the threshold can be chosen to optimise the balance between sensitivity and specificity. Algorithm 1: first algorithm, Algorithm B: second algorithm.

TABLE 6
Analyses of a set of 94 CRC patients
and 172 normal control individuals.

| quantitation threshold (ng/ml) | sensitivity | specificity | accuracy |
|---|---|---|---|
| 2 out of 3 (Algorithm A) | 0.553 | 0.977 | 0.827 |
| 0.5 | 0.553 | 0.977 | 0.827 |
| 1 | 0.553 | 0.971 | 0.823 |
| 2 | 0.585 | 0.965 | 0.831 |
| 3 | 0.617 | 0.965 | 0.842 |
| 4 | 0.66 | 0.953 | 0.85 |
| 5 | 0.67 | 0.948 | 0.85 |
| 6 | 0.691 | 0.936 | 0.85 |
| 7 | 0.702 | 0.924 | 0.846 |
| 8 | 0.723 | 0.919 | 0.85 |
| 9 | 0.734 | 0.913 | 0.85 |
| 10 | 0.734 | 0.913 | 0.85 |
| 11 | 0.745 | 0.907 | 0.85 |
| 12 | 0.745 | 0.907 | 0.85 |
| 13 | 0.745 | 0.901 | 0.846 |
| 14 | 0.745 | 0.901 | 0.846 |
| 15 | 0.745 | 0.895 | 0.842 |
| 16 | 0.745 | 0.895 | 0.842 |
| 17 | 0.745 | 0.89 | 0.838 |
| 18 | 0.745 | 0.89 | 0.838 |
| 19 | 0.745 | 0.884 | 0.835 |
| 20 | 0.745 | 0.884 | 0.835 |
| 25 | 0.745 | 0.884 | 0.835 |
| 30 | 0.745 | 0.878 | 0.831 |
| 50 | 0.745 | 0.872 | 0.827 |
| 1 out of 3 (Algorithm B) | 0.745 | 0.866 | 0.823 |

Column 1 shows examples of set thresholds of a CCF1 quantitation of DNA extracted from plasma. Column 2 provides the respective sensitivity estimates. Column 3 provides the respective specificity estimates. Column 4 provides the respective accuracy estimates. Highlighted (bold) are exemplary thresholds that provide significant better performance, when the concentration dependent algorithm is applied. Depending on the main focus of the clinical application the threshold can be chosen to optimise the balance between sensitivity and specificity. Algorithm 1: first algorithm, Algorithm B: second algorithm.

### REFERENCES OF THE EXAMPLES

1. Levin B, Lieberman D A, McFarland B, Smith R A, Brooks D, Andrews K S, et al. Screening and surveillance for the early detection of colorectal cancer and adenomatous polyps, 2008: a joint guideline from the American Cancer Society, the US Multi-Society Task Force on Colorectal Cancer, and the American College of Radiology. CA Cancer J Clin 2008; 58:130-60.
2. Gupta A K, Brenner D E, Turgeon D K. Early detection of colon cancer: new tests on the horizon. Mol Diagn Ther 2008; 12:77-85.
3. Diehl F, Schmidt K, Durkee K H, Moore K J, Goodman S N, Shuber A P, et al. Analysis of mutations in DNA isolated from plasma and stool of colorectal cancer patients. Gastroenterology 2008; 135:489-98.
4. Leon S A, Shapiro B, Sklaroff D M, Yaros M J. Free DNA in the serum of cancer patients and the effect of therapy. Cancer Res 1977; 37:646-50.
5. Stroun M, Anker P, Maurice P, Lyautey J, Lederrey C, Beljanski M. Neoplastic characteristics of the DNA found in the plasma of cancer patients. Oncology 1989; 46:318-22.
6. Sorenson G D, Pribish D M, Valone F H, Memoli V A, Bzik D J, Yao S L. Soluble normal and mutated DNA sequences from single-copy genes in human blood. Cancer Epidemiol Biomarkers Prey 1994; 3:67-71.
7. Esteller M, Sanchez-Cespedes M, Rosell R, Sidransky D, Baylin S B, Herman J G. Detection of aberrant promoter hypermethylation of tumor suppressor genes in serum DNA from non-small cell lung cancer patients. Cancer Res 1999; 59:67-70.
8. Wong I H, Lo Y M, Zhang J, Liew C T, Ng M H, Wong N, et al. Detection of aberrant p16 methylation in the plasma and serum of liver cancer patients. Cancer Res 1999; 59:71-3.
9. Grady W M, Carethers J M. Genomic and Epigenetic Instability in Colorectal Cancer Pathogenesis. Gastroenterology 2008.
10. Chan T A, Glockner S, Yi J M, Chen W, Van Neste L, Cope L, et al. Convergence of mutation and epigenetic alterations identifies common genes in cancer that predict for poor prognosis. PLoS Med 2008; 5:e114.
11. Lofton-Day C, Model F, Devos T, Tetzner R, Distler J, Schuster M, et al. DNA methylation biomarkers for blood-based colorectal cancer screening. Clin Chem 2008; 54:414-23.
12. Diehl F, Li M, Dressman D, He Y, Shen D, Szabo S, et al. Detection and quantification of mutations in the plasma of patients with colorectal tumors. Proc Natl Acad Sci USA 2005; 102:16368-73.
13. Grutzmann R, Molnar B, Pilarsky C, Habermann J K, Schlag P M, Saeger H D, et al. Sensitive detection of colorectal cancer in peripheral blood by Septin 9 DNA methylation assay. Plos One 2008; In Press.
14. Chan K C, Lai P B, Mok T S, Chan H L, Ding C, Yeung S W, Lo Y M. Quantitative analysis of circulating methylated DNA as a biomarker for hepatocellular carcinoma. Clin Chem 2008; 54:1528-36.
15. Tong Y K, Lo Y M. Diagnostic developments involving cell-free (circulating) nucleic acids. Clin Chim Acta 2006; 363:187-96.
16. Tetzner R, Dietrich D, Distler J. Control of carry-over contamination for PCR based DNA methylation quantification using bisulfite treated DNA. Nucleic Acids Res 2007; 35:e4.
17. Rasmussen R. Quantification on the LightCycler. In: Meuer S, Wittwer, C., and Nakagawara, K., ed. Rapid Cycle Real-time PCR, Methods and Applications, Vol. Heidelberg: Springer Press, 2001:21-34.
18. Chiu R W, Poon L L, Lau T K, Leung T N, Wong E M, Lo Y M. Effects of blood processing protocols on fetal and total DNA quantification in maternal plasma. Clin Chem 2001; 47:1607-13.
19. Cottrell S E, Distler J, Goodman N S, Mooney S H, Kluth A, Olek A, et al. A real time PCR assay for DNA-methylation using methylation-specific blockers. Nucleic Acids Res 2004; 32:e10.
20. Tong Y K, Ding C, Chiu R W, Gerovassili A, Chim S S, Leung T Y, et al. Noninvasive prenatal detection of fetal trisomy 18 by epigenetic allelic ratio analysis in maternal plasma: Theoretical and empirical considerations. Clin Chem 2006; 52:2194-202.
21. Grunau C, Hindermann W, Rosenthal A. Large-scale methylation analysis of human genomic DNA reveals tissue-specific differences between the methylation profiles of genes and pseudogenes. Hum Mol Genet 2000; 9:2651 63.
22. Tong Y K, Lo Y M. Plasma epigenetic markers for cancer detection and prenatal 631 diagnosis. Front Biosci 2006; 11:2647-56.
23. Grunau C, Clark S J, Rosenthal A. Bisulfite genomic sequencing: systematic investigation of critical experimental parameters. Nucleic Acids Res 634 2001; 29:E65-5.
24. Guadalajara H, Dominguez-Berzosa C, Garcia-Arranz M, Herreros M D, Pascual I, Sanz-Baro R, et al. The concentration of deoxyribonucleic acid in plasma from 73 patients with colorectal cancer and apparent clinical correlations. Cancer Detect Prey 2008; 32:39-44.
25. Diehl F, Schmidt K, Choti M A, Romans K, Goodman S, Li M, et al. Circulating mutant DNA to assess tumor dynamics. Nat Med 2008; 14:985-90.
26. Boni L, Cassinotti E, Canziani M, Dionigi G, Rovera F, Dionigi R. Free circulating DNA as possible tumour marker in colorectal cancer. Surg Oncol 2007; 16 Suppl 1:S29-31.
27. Bennett K L, Karpenko M, Lin M T, Claus R, Arab K, Dyckhoff G, et al. Frequently methylated tumor suppressor genes in head and neck squamous cell carcinoma. Cancer Res 2008; 68:4494-9.
28. Burrows J F, Chanduloy S, Mcllhatton M A, Nagar H, Yeates K, Donaghy P, et al. Altered expression of the septin gene, SEPT9, in ovarian neoplasia. J Pathol 2003; 201:581-8.
29. Morikawa T, Kato J, Yamaji Y, Wada R, Mitsushima T, Shiratori Y. A comparison of the immunochemical fecal occult blood test and total colonoscopy in the asymptomatic population. Gastroenterology 2005; 129: 422-8.
30. Allison J E, Sakoda L C, Levin T R, Tucker J P, Tekawa I S, Cuff T, et al. Screening for colorectal neoplasms with new fecal occult blood tests: update on performance characteristics. J Natl Cancer Inst 2007; 99: 1462-70.
31. Ahlquist D A, Sargent D J, Loprinzi C L, Levin T R, Rex D K, Ahnen D J, et al. Stool DNA and occult blood testing for screen detection of colorectal neoplasia. Ann Intern Med 2008; 149: 441-50, W81.

1. A method for validating an assay for determining the presence or absence of a medical condition based on the methylation status of a nucleic acid in a biological sample, wherein the nucleic acid has been treated such that all unmethylated cytosine bases are converted to uracil bases, comprising the following steps:
measuring the concentration of the nucleic acid in a multitude of biological samples;
allotting the samples based on the measured concentration of the nucleic acid in the samples
to a first sample group if the measured concentration of the nucleic acid is below a given threshold value, or
to a second sample group if the measured concentration of the nucleic acid is at or above the given threshold value;
performing an assay for determining the methylation status of the nucleic acid in the sample at least twice for obtaining at least two methylation values;
applying
a first algorithm to the value of the samples of the first sample group for determining a methylation result of the assay, or
a second algorithm to the value of the samples of the second sample for determining a methylation result of the assay; and
validating the assay by determining whether sensitivity and specificity of the assay reach given values.

2. The method according to claim 1, wherein the threshold value is chosen such that a first fraction of the samples is allotted to the first sample group and a second fraction of the samples is allotted to the second sample group.

3. The method according to claim 1, wherein the samples are also allotted based on the measured concentration of the nucleic acid in the sample to
a third sample group if the measured concentration of the nucleic acid is below a given minimum threshold value, wherein the samples allotted to this third sample group are not used for assay validation.

4. The method according to claim 1, wherein the biological sample stems from a body fluid.

6. The method according to claim 1, wherein the nucleic acid is genomic DNA.

7. The method according to claim 1, wherein chemical reagent is bisulfite.

8. The method according to claim 1, wherein determining the presence or absence of methylation is determined by means of an assay taken from the group comprising array based assays, real-time assays, MSP, MethyLight, QM, and HeavyMethyl.

9. The method according to claim 1, wherein the target nucleic acid comprises a nucleic acid region comprising at least one CpG that can be methylated.

10. The method according to claim 1, wherein the target nucleic acid is a gene, including a regulatory sequence or promoter, preferably wherein the target nucleic acid gene is the Septin-9 gene or a fragment thereof.

11. The method according to claim 1 wherein the medical condition is selected from the group consisting of cancers, solid tumors and cell proliferative disorders.


SEQUENCE LISTING (12 sequences)

SEQ ID NO: 1 - DNA, 91 bp, Homo sapiens
  bisulfite treated
cccaccaaccatcatatcgaaccccgcgatcaacgcgcaactaaataaaatcatttcgaacttcgaaaataaatactaaactaactactac

SEQ ID NO: 2 - DNA, 65 bp, Homo sapiens
  bisulfite treated
gattngttgtttattagttattatgtcggatttcgcggttaacgcgtagttggatgggattattt

SEQ ID NO: 3 - DNA, 19 bp, Artificial
  Primer
aaataatcccatccaacta

SEQ ID NO: 4 - DNA, 27 bp, Artificial
  Primer
gattdsgttgtttattagttattatgt

SEQ ID NO: 5 - DNA, 33 bp, Artificial
  Completely synthesized blocker
gttattatgttggattttgtggttaatgtgtag

SEQ ID NO: 6 - DNA, 18 bp, Artificial
  Probe
ttaaccgcgaaatccgac

SEQ ID NO: 7 - DNA, 25 bp, Artificial
  Primer
taagagtaataatggatggatgatg

SEQ ID NO: 8 - DNA, 17 bp, Artificial
  Primer
cctcccatctcccttcc

SEQ ID NO: 9 - DNA, 25 bp, Artificial
  Probe
atggatgaagaaagaaaggatgagt

SEQ ID NO: 10 - DNA, 24 bp, Artificial
  Primer
gtgatggaggaggtttagtaagtt

SEQ ID NO: 11 - DNA, 25 bp, Artificial
  Primer
ccaataaaacctactcctcccttaa

SEQ ID NO: 12 - DNA, 30 bp, Artificial
  Probe
accaccacccaacacacaataacaaacaca